(19) United States
(12) Patent Application Publication
(10) Pub. No.: US 2007/0110216 A1
(43) Pub. Date: May 17, 2007

(54) Product to tailor distributions of energy deposition by a high energy photon beam

(76) Inventor: Leonard Reiffel, Chicago, IL (US)

Correspondence Address: William J Hallihan, 217 N Jefferson Street, Suite 100, Chicago, IL, 60661, US

(21) Appl. No.: 10/573,715

(22) PCT Filed: Sep. 27, 2004
(86) PCT No.: PCT/US04/31699

§ 371 Date: Mar. 28, 2006

(51) Int. Cl. A61N 5/10; H05G 2/00; G21G 4/00; H01J 35/00

(52) U.S. Cl. 378/119; 378/65

(57) ABSTRACT

A product tailors energy deposition. In one form, the product includes a magnetic field. The magnetic field is in a pre-set tailoring relationship with a body, a target volume in the body, and a electron-photon cascade in the body produced by a photon beam, where the photon beam and the electron-photon cascade are substantially parallel to a beam path. In one form, the magnetic field has a component non-parallel to the beam path in the target volume, which is at least one hundred gauss. The target volume having a target density and the body having a body density proximal the target volume. In one form, the tailoring relationship causes a desired distribution of energy deposited in the body and the target volume.

This application claims priority benefit of U.S. provisional application No. 60/506,792 filed Sep. 29, 2003, which is incorporated herein by reference.

The product uses magnetic fields and density differences to tailor distributions of energy deposition caused by a high energy photon beam. For example, the product can combine a magnetic field with a difference in density between a target volume in a body and the body proximal the target volume to increase energy deposition in a target volume in a body relative to parts of the body other than the target volume.

Though the product can be useful in radiation therapy for cancer, the product can also be used in various other applications, such as in materials technologies, where it can be useful to tailor distributions of energy deposition by a high energy photon beam.

The product results from unexpected discoveries made in research with the product claimed in U.S. Pat. No. 05,974,112 (also referred to as U.S. Pat. No. 5,974,112), issued 26 Oct. 1999, which is incorporated herein by reference.

The product taught in U.S. Pat. No. 05,974,112 comprises a magnetic field having a high gradient along a high energy photon beam path. As the photon beam enters a body, high energy electrons are produced by the photons via scattering and pair production. Thus, an electron-photon cascade proceeds along the beam path with the electrons losing energy to the body.

When the electrons enter the high field region along the magnetic field gradient, the Lorentz force on the electrons causes these electrons to lose more energy in this region then would be the case without the magnetic field. Thus, when the high field region is at a target volume in the body, more energy is deposited in the target volume than would otherwise be the case and, since electrons have been thereby removed from the beam, less energy is deposited in a region beyond the target volume than would otherwise be the case.

Unexpectedly, in research with this, other ways have been found to tailor a relationship with a body, a target volume in the body, a magnetic field, and an electron-photon cascade in the body produced by a photon beam, where the photon beam and the electron-photon cascade proceed substantially parallel to a beam path.

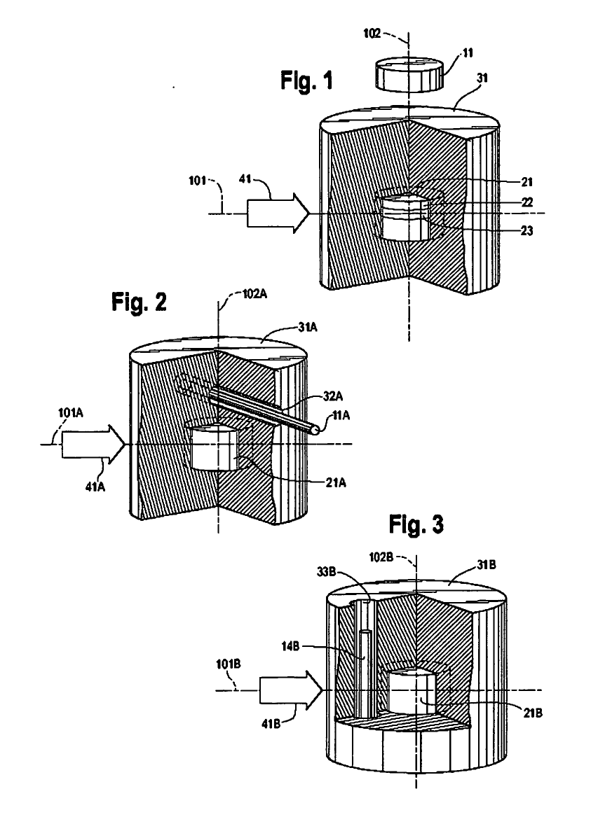

FIG. 1 shows a magnetic field source 11 proximal a body with a target volume in the body irradiated by a photon beam 41.

FIG. 2 shows a magnetic field source 11A inserted in a cavity in a body proximal a target volume in the body irradiated by a photon beam.

FIG. 3 shows a inserted volume in the body between the photon beam and the target volume. A source of the magnetic field is not shown here as the source could be an external source as in FIG. 1 and could be an internal source as in FIG. 2 and could be combinations of these.

The product to tailor energy deposition comprises a magnetic field which is in a pre-set tailoring relationship with a body 31, 31A, 31B; a target volume 21, 21A, 21B in the body; and a electron-photon cascade in the body produced by a photon beam 41, 41A, 41B; where the photon beam and the electron-photon cascade proceed substantially parallel to a beam path which is centered on a beam axis 101, 101A, 101B, and can be non-parallel to a field axis 102, 102A, 102B. In one form, the photon beam and the electron-photon cascade proceed substantially parallel to a beam path which is centered on a beam axis 101, 101A, 101B, and is orthogonal to a field axis 102, 102A, 102B.

It is believed that in most, if not all, forms of the product the magnetic field has a component in the target volume parallel to the field axis of at least one hundred gauss.

The target volume has a target density. The body has a body density proximal the target volume.

The magnetic field can have a gradient along the beam axis, and need not have this gradient.

The magnetic field can be produced by any magnetic field source and by any combinations of magnetic field sources.

The tailoring relationship causes a desired distribution of energy deposited in the body and the target volume.

In a form of the product, where the target density is least thirty percent greater than the body density, the preset tailoring relationship can also comprise a component of the magnetic field in the body proximal the target volume parallel to the field axis, of at least one hundred gauss.

This magnetic field tailors the energy deposition to be higher in the target volume than it would be without the magnetic field. Unlike the case in the issued patent U.S. Pat. No. 05,974,112, referenced above, this magnetic field need not have a high gradient along the beam path.

In this form of the product the magnetic field can be produced by a magnetic field source inserted 11A in a cavity 32A in the body.

In all forms of the invention the magnetic field source can be an external source as indicated in FIG. 1, can be several external sources, can be an internal source as indicated in FIG. 2, can be several internal sources, and can be various combinations of these.

In this form of the product the inserted magnetic field source can be chosen from a plurality of graded magnetic field sources in order to match the cavity and desired the tailoring relationship.

In a form of the product the tailoring relationship can comprise an inserted volume 14B inserted in a cavity 33B in the body between the photon beam and the target volume and also comprise a component of the magnetic field parallel to the field axis axis in the inserted volume, of at least one hundred gauss, where the inserted volume differs in density by at least twenty percent from the body density.

The inserted volume can have a density at least twenty percent less than the target volume density. In this case the combination of the magnetic field and the inserted volume will tailor the energy deposition to have a lower value just after the inserted volume, than would be the case without the inserted volume.

The inserted volume can have a density at least twenty percent more than the target volume density. In this case the combination of the magnetic field and the inserted volume will tailor the energy deposition to have a higher value just beyond the inserted volume, than would be the case without the inserted volume.

In a form of the product when there is a magnetic field gradient along the field axis, then the tailoring relationship can comprise sizing a first exposure of the photon beam so that it irradiates a first slice 22 of the target volume parallel to the photon beam, and sizing a second exposure of the photon beam so that it irradiates a second slice 23 of the target volume parallel to the photon beam, where the magnetic field is changed to be substantially the same in the second slice during the second exposure as in the first slice during the first exposure.

In this form of the product the magnetic field can be changed for the second exposure by moving a magnetic field source 11 along axis 102.

Alternatively, in this form of the product, the tailoring relationship can comprise a photon beam energy gradient parallel to the field axis 102, which matches the magnetic field gradient so that substantially the same energy is deposited in a first slice 22 of the target volume parallel to the photon beam as in a second slice 23 of the target volume parallel to the photon beam.

In all forms of the product a magnetic field source can be chosen from a plurality of magnetic field sources.

One magnetic field source from the plurality of magnetic field sources can be the external magnetic field source depicted in FIG. 1. Another magnetic field source from the plurality of magnetic field sources can be the external magnetic field source depicted in FIG. 2.

A first magnetic field source from the plurality of magnetic field sources can have a first magnetic field configuration. A second magnetic field source from the plurality of magnetic field sources can have a second magnetic field configuration which is different from the first magnetic field configuration. An ith magnetic field source from the plurality of magnetic field sources can have an ith magnetic field configuration which is different from the first magnetic field configuration and different from the first magnetic field configuration.

At least one magnetic field source from the plurality of magnetic field sources can be a superconducting magnet.

At least one magnetic field source from the plurality of magnetic field sources can have any power and cooling needs attached via a flexible conduit.

At least one magnetic field source from the plurality of magnetic field sources can comprise a configuration of several electromagnets.

At least one magnetic field source from the plurality of magnetic field sources can have a face which is warmed only temporarily for use.

What is claimed is:

1. A product to tailor energy deposition, the product comprising:
a magnetic field, the magnetic field being in a pre-set tailoring relationship with a body, a target volume in the body, and a electron-photon cascade in the body produced by a photon beam, where the photon beam and the electron-photon cascade are substantially parallel to a beam path,
the magnetic field having a component non-parallel to the beam path in the target volume, which is at least one hundred gauss;
the target volume having a target density;
the body having a body density proximal the target volume; and
the tailoring relationship causing a desired distribution of energy deposited in the body and the target volume.

2. The product of claim 1 wherein:
the target density is at least thirty percent greater than the body density; and
the preset tailoring relationship comprises a component of the magnetic field in the body proximal the target volume, non-parallel to the beam path of at least one hundred gauss.

3. The product of claim 2 wherein the magnetic field is produced by a magnetic field source inserted in a cavity in the body.

4. The product of claim 3 wherein the inserted magnetic field source is chosen from a plurality of graded magnetic field sources in order to match the cavity and the tailoring relationship.

5. The product of claim 1 wherein the tailoring relationship comprises:
an inserted volume,
the inserted volume differing in density by at least twenty percent from the body density, and
the magnetic field having a component in the inserted volume, non-parallel to the beam path of at least one hundred gauss.

6. The product of claim 1 wherein there is a magnetic field gradient non-parallel to the beam path, and wherein the tailoring relationship comprises:
sizing a first exposure of the photon beam so that it irradiates a first slice of the target volume parallel to the photon beam; and
sizing a second exposure of the photon beam so that it irradiates a second slice of the target volume parallel to the photon beam, the magnetic field having been changed to be substantially the same in the second slice during the second exposure as in the first slice during the first exposure.

7. The product of claim 6 wherein the magnetic field is changed for the second exposure by moving a magnetic field source.

8. The product of claim 6 wherein the tailoring relationship comprises a photon beam energy gradient non-parallel to the photon beam which matches the magnetic field gradient so that substantially the same energy is deposited in a first slice of the target volume parallel to the photon beam as in a second slice of the target volume parallel to the photon beam.

9. The product of claim 1 wherein the magnetic field is provided by a magnetic field source chosen from a plurality of magnetic field sources wherein:
a first magnetic field source chosen from the plurality of magnetic field sources has a first magnetic field configuration; and
a second magnetic field source from the plurality of magnetic field sources has a second magnetic field configuration which is different from the first magnetic field configuration.

10. The product of claim 9 wherein at least one magnetic field source from the plurality of magnetic field sources is a superconducting magnet.

11. The product of claim 9 wherein at least one magnetic field source from the plurality of magnetic field sources has power and cooling needs attached via a flexible conduit.

12. The product of claim 9 wherein at least one magnetic field source from the plurality of magnetic field sources comprises a configuration of several electromagnets.

13. The product of claim 9 wherein at least one magnetic field source from the plurality of magnetic field sources has a face which is warmed only temporarily for use.

14. A method of tailoring energy deposition, the method comprising:
providing a target volume having a target density;
providing a body having a body density proximal the target volume, wherein the target volume is in the body;
providing a magnetic field;
tailoring the magnetic field in a relationship with the body, the target volume, and a electron-photon cascade in the body produced by a photon beam, where the photon beam and the electron-photon cascade are substantially parallel to a beam path, wherein the magnetic field has a component non-parallel to the beam path in the target volume, which is at least one hundred gauss; and
the tailoring relationship causing a desired distribution of energy deposited in the body and the target volume.

15. The method of claim 14 wherein the magnetic field has a component orthogonal to the beam path in the target volume.

16. The method of claim 14 further comprising inserting a magnetic field source inserted in a cavity in the body to produce the magnetic field.

17. The method of claim 16 further comprising choosing the magnetic field source from a plurality of graded magnetic field sources in order to match the cavity and the tailoring relationship.

18. The method of claim 14 further comprising inserting a volume in the body, the volume differing in density by at least twenty percent from the body density, wherein the magnetic field has a component in the inserted volume, non-parallel to the beam path of at least one hundred gauss.

19. The method of claim 14 further comprising:
providing a magnetic field gradient non-parallel to the beam path, and wherein tailoring the magnetic field comprises:
sizing a first exposure of the photon beam so that it irradiates a first slice of the target volume parallel to the photon beam; and
sizing a second exposure of the photon beam so that it irradiates a second slice of the target volume parallel to the photon beam, the magnetic field having been changed to be substantially the same in the second slice during the second exposure as in the first slice during the first exposure.

20. The method of claim 19 wherein tailoring the magnetic field comprises matching a photon beam energy gradient non-parallel to the photon beam to the magnetic field gradient so that substantially the same energy is deposited in a first slice of the target volume parallel to the photon beam as in a second slice of the target volume parallel to the photon beam.
